SEQUENCE LISTING (7 sequences)

SEQ ID NO: 1 - RNA, 22 nt, Homo sapiens
cgcauccccuagggcauuggug

SEQ ID NO: 2 - RNA, 23 nt, Homo sapiens
cccacugccccaggugcugcugg

SEQ ID NO: 3 - RNA, 21 nt, Homo sapiens
cuuuuugcggucugggcuugc

SEQ ID NO: 4 - RNA, 22 nt, Homo sapiens
aagcccuuaccccaaaaaguau

SEQ ID NO: 5 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 6 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 7 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

(19) United States
(12) Patent Application Publication
(10) Pub. No.: US 2022/0211741 A1
(43) Pub. Date: Jul. 7, 2022

(54) METHODS FOR THE TREATMENT AND PROGNOSIS OF CANCER

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); UNIVERSITÉ PARIS-EST CRÉTEIL VAL DE MARNE, Creteil (FR)

(72) Inventors: Fouad LAFDIL, Creteil (FR); Jean-Michel PAWLOTSKY, Creteil (FR); Camilia MACHOU, Creteil (FR)

(21) Appl. No.: 17/604,510

(22) PCT Filed: Apr. 17, 2020
(86) PCT No.: PCT/EP2020/060888

§ 371 Date: Oct. 18, 2021

(30) Foreign Application Priority Data
Apr. 18, 2019 (EP) 19305507.6
Jul. 18, 2019 (EP) 19305955.7

(51) Int. Cl. A61K 31/713; C12Q 1/6886; A61P 35/00; A61K 45/06

(52) U.S. Cl. CPC A61K 31/713; A61K 45/06; A61P 35/00; C12Q 1/6886

(57) ABSTRACT

Cancer is a group of diseases involving abnormal cell growth with the potential to invade or spread to other parts of the body. In particular, hepatocellular carcinoma (HCC) has become the most common primary hepatic malignancy. Current therapies are now satisfying and there is therefore an important need for identifying new therapeutic avenues. IL-27 is a cytokine produced in liver microenvironment but its role in the pathogenesis of HCC has never been investigated. The inventors now show that IL-27 exerts anti-proliferative activities in HCC cell lines. However, the inventors show that in patients suffering from HCC that a decreased expression of WSX-1 (i.e. the IL-27 receptor) is associated with a worse prognosis and contributes to the tumor proliferation. The inventors then identified some microRNAs (miR) that are capable of repressing the expression of WSX-1 and show that overexpression of said miR are associated with a worse prognosis in patients. Finally, the inventors demonstrate that antagomirs restore the expression of WSX-1 that can thus restore the tumor cell sensitization to IL-27 properties. Accordingly, the present invention relates to methods for the treatment and prognosis of cancer, in particular hepatocellular carcinoma (HCC).

### FIELD OF THE INVENTION

The present invention relates to methods for the treatment and prognosis of cancer, in particular hepatocellular carcinoma.

### BACKGROUND OF THE INVENTION

Cancer is a group of diseases involving abnormal cell growth with the potential to invade or spread to other parts of the body.

In particular, hepatocellular carcinoma (HCC) has become the most common primary hepatic malignancy. It now ranks sixth in the world among all malignancies, contributing to the third leading cause of mortality attributed to cancer worldwide. Despite significant progress in HCC diagnosis and improvement of the curative strategies, the HCC incidence has been continuously increasing since the 1970's, being multiplied by 3-fold. HCC remains a life-threatening disease, with a 5-year survival rate of less than 10% in western countries. The major risk factors of HCC are chronic infections with hepatitis B or hepatitis C virus (HBV or HCV, respectively). Chronic hepatitis can progress into cirrhosis (a noncancerous liver disease associated with fibrosis and abnormal nodules), which increases the risk of developing HCC. Advanced age, being male, obesity, alcohol abuse, diabetic, and family history, are also variables associated with increased risks for developing HCC. The developed countries have increasingly seen non-alcoholic steatohepatitis (NASH) as a primary contributor for HCC. It is assumed that the obesity epidemic and prevalence of diabetes has played a significant role.

Current management of HCC includes surgical resection/hepatectomy, liver transplantation (deceased and living), thermal or chemical ablation, chemoembolization, and medical treatment. The pathophysiologic complexity of HCC progression has made medical treatment of HCC challenging. It has been difficult to provide adequate tumor therapy but at the same time maintaining liver function and patient's general conditions. Sorafenib, which is an oral tyrosine kinase inhibitor, was the gold standard treatment option for advanced HCC but demonstrated mild improvement in survival rate for progressive HCC with an increased median survival of only 3 months. However, the number of patients that did not respond to Sorafenib are still high and side effects are significant. Currently, a novel kinase inhibitor, Lenvatinib that underwent a phase 3 randomised controlled trial in a cohort of patients with advanced HCC, is also proposed and showed non-inferiority in terms of overall survival when compared to Sorafenib. There is no second line agent available and there is therefore a need to find new therapeutic avenues. The pathophysiology of HCC is an evolving topic and appears to be multifactorial. HCC predominantly arises in a cirrhotic liver where repeated inflammation occurs along with fibrogenesis, which predispose subsequently the liver to malignant transformation. Thus, the inflammatory microenvironment plays a prominent part in starting the advancement towards HCC. In the last decade several immune cells and cytokines in the microenvironment of several types of cancers have been described with anti- or pro tumoral properties.

Among those cytokines, IL-27 which is a two-chain cytokine, composed of EBI3 and IL-27p28 subunits belongs to the IL-12 family and signals through its heterodimeric receptor composed of gp130 and IL-27 receptor alpha (WSX-1) subunits. Several pieces of evidence, obtained in preclinical tumor models, indicated that IL-27 has a potent antitumor activity, related not only to the induction of tumor-specific Th1 and cytotoxic T lymphocyte (CTL) responses but also to direct inhibitory effects on tumor cell proliferation, survival, invasiveness, and angiogenic potential (Fabbi M et al. Mediators Inflamm. 2017; 2017:3958069). Nonetheless, given its immune-regulatory functions, the effects of IL-27 on cancer may be dual and protumor effects may also occur (Fabbi M et al. Mediators Inflamm. 2017; 2017:3958069). Its role in HCC has never been investigated. The literature only reports that IL-27 is mainly expressed in liver by immune and epithelial cells, including hepatocytes contributing to liver regeneration (Guillot A. et al. Hepatol Commun. 2018 Jan. 30; 2(3):329-343).

### SUMMARY OF THE INVENTION

As defined by the claims, the present invention relates to methods for the treatment and prognosis of cancer.

### DETAILED DESCRIPTION OF THE INVENTION

Cancer is a group of diseases involving abnormal cell growth with the potential to invade or spread to other parts of the body. In particular, hepatocellular carcinoma (HCC) has become the most common primary hepatic malignancy. Despite significant progress in HCC therapeutic options and diagnosis, there is still an important need for identifying more effective therapeutic avenues. IL-27 is a cytokine produced in liver microenvironment but its role in the pathogenesis of HCC has never been investigated. The inventors now show that IL-27 exerts anti-proliferative activities in HCC cell lines. However, the inventors show that in patients suffering from HCC that a decreased expression of WSX-1 (i.e. the IL-27 receptor) is associated with a worse prognosis and contributes to the tumor cell proliferation. The inventors then identified some microRNAs (miR) that are capable of repressing the expression of WSX-1 and show that overexpression of said miR are associated with a worse prognosis in patients. Finally, the inventors demonstrate that selective antagomirs restore the expression of WSX-1 that can thus restore the tumor cell sensitization to IL-27 properties.

### Methods for the Treatment of Cancer

Accordingly, the first object of the present invention relates to a method of treating cancer in patient in need thereof comprising administering to the patient a therapeutically effective amount of a miR-324 inhibitor and/or miR-129 inhibitor.

As used herein, the term “cancer” has its general meaning in the art and includes, but is not limited to, hematopoietic cancers (e.g. blood borne tumors) and non-hematopoietic cancers (e.g. solid tumors). The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, tumor cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the method of the present invention is particularly suitable for the treatment of hepatocellular carcinoma. As used herein, the term “hepatocellular carcinoma” or “HCC” has its general meaning in the art and refers to a malignant tumor of hepatocellular origin that may develop in patients with risk factors that include alcohol abuse, viral hepatitis, and metabolic liver disease. HCC is a type of liver cancer. HCC can undergo hemorrhage and necrosis because of a lack of fibrous stroma. Vascular invasion, particularly of the portal system, is common. Aggressive HCC can cause hepatic rupture and hemoperitoneum.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, increasing the quality of life, and/or prolonging survival. Also encompassed by “treatment” is a reduction of pathological consequence of cancer. The methods of the present invention contemplate any one or more of these aspects of treatment. In some embodiments, the terms “treating”, or “treatment” refers to both therapeutic treatment and prophylactic or preventative measures; wherein the object is to prevent or slow down (lessen) the targeted disease. Therefore, in some embodiments, those in need of treatment may include those already with the disorder as well as those prone to have the disorder or those in whom the disorder is to be prevented.

As used herein, the term “microRNA” or “miRNA” refers to an RNAi agent that is approximately 21-23 nucleotides (nt) in length. miRNAs can range between 18-26 nucleotides in length. Typically, miRNAs are single-stranded. However, in various embodiments, miRNAs may be at least partially double-stranded. In certain embodiments, miRNAs may comprise an RNA duplex (referred to herein as a “duplex region”) and may optionally further comprises one or two single-stranded overhangs. In various embodiments, an RNAi agent comprises a duplex region ranging from 15 to 29 by in length and optionally further comprising one or two single-stranded overhangs. A miRNA may be formed from two RNA molecules that hybridize together or may alternatively be generated from a single RNA molecule that includes a self-hybridizing portion. In general, free 5′ ends of miRNA molecules have phosphate groups, and free 3′ ends have hydroxyl groups. The duplex portion of a miRNA usually, but does not necessarily, comprise one or more bulges consisting of one or more unpaired nucleotides. One strand of a miRNA includes a portion that hybridizes with a target RNA. In certain embodiments of the invention, one strand of the miRNA is not precisely complementary with a region of the target RNA, meaning that the miRNA hybridizes to the target RNA with one or more mismatches. In other embodiments of the invention, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in various embodiments, miRNAs may mediate inhibition of gene expression by causing degradation of target transcripts.

As used herein, the term “miR-324” has its general meaning in the art and refers to the miR available from the data base http://mirbase.org under the miRBase accession number MI0000813 (hsa-mir-324). The term encompasses the mature sequences hsa-miR-324-5p (MIMAT0000761, SEQ ID NO:1) and hsa-miR-324-3p (MIMAT0000762, SEQ ID NO:2).

>hsa-miR-324-5p MIMAT0000761
SEQ ID NO: 1
CGCAUCCCCUAGGGCAUUGGUG
>hsa-miR-324-3p MIMAT0000762
SEQ ID NO: 2
CCCACUGCCCCAGGUGCUGCUGG

As used herein, the term “miR-129” has its general meaning in the art and refers to the miR available from the data base http://mirbase.org under the miRBase accession number MI0000252 (hsa-miR-129-1). The term encompasses the mature sequences hsa-miR-129-5p (MIMAT0000242, SEQ ID NO:3) and hsa-miR-129-1-3p (MIMAT0004548, SEQ ID NO:4).

>hsa-miR-129-5p MIMAT0000242
SEQ ID NO: 3
CUUUUUGCGGUCUGGGCUUGC
>hsa-miR-129-1-3p MIMAT0004548
SEQ ID NO: 4
AAGCCCUUACCCCAAAAAGUAU

As used herein, the term “miR inhibitor compound” refers to any compound able to prevent the action of miR-324 or miR-129. The miR inhibitor compound of the present invention is a compound that inhibits or reduces the activity of miR-324 or miR-129. However, decreasing and/or reducing the activity of miR-324 or miR-129 can also be obtained by inhibiting miR-324 or miR-129 expression. The term “inhibiting miR-324 or miR-129 expression” means that the production of miR-324 or miR-129 in tumor cells after treatment is less than the amount produced prior to treatment or neutralize the activity of existent amount. One skilled in the art can readily determine whether miR-324 or miR-129 expression has been inhibited in the tumor cells, using for example the techniques for determining miRNA transcript level.

In some embodiments, the miR inhibitor compound of the invention is a compound such as nucleic acid that hybridizes with miR-324 or miR-129 or having sequence complementarity to that of miR-324 or miR-129. In some embodiments, miR inhibitor compound of the invention is a compound such as nucleic acid having at least 80, 81, 82, 83, 84, 85, 86, 87, 88, 89, 90, 91, 92, 93, 94, 95, 96, 97, 98 99 or 100% sequence complementarity to that of miR-324 or miR-129.

Suitable miR inhibitor compounds include double-stranded RNA (such as short- or small-interfering RNA or “siRNA”), antagomirs, antisense nucleic acids, and enzymatic RNA molecules such as ribozymes. Each of these compounds can be targeted to a given miRNA and destroy or induce the destruction of the target miRNA. For example, expression of a given miRNA can be inhibited by inducing RNA interference of the miRNA with an isolated double-stranded RNA (“dsRNA”) molecule which has at least 90%, for example 90%; 91%, 92%, 93%, 94%, 95%, 96%, 97%, 98%, 99% or 100%, sequence homology with at least a portion of the miRNA. In some embodiments, the dsRNA molecule is a “short or small interfering RNA” or “siRNA”.

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target miRNA.

As used herein, a nucleic acid sequence in a siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area. The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA can also comprise a 3 overhang. As used herein, a “3′ overhang” refers to at least one unpaired nucleotide extending from the 3′-end of a duplexed RNA strand. Thus, in some embodiments, the siRNA comprises at least one 3′ overhang of 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In some embodiments, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above. Exemplary methods for producing and testing dsRNA or siRNA molecules are described in U.S. published patent application 2002/0173478 to Gewirtz and in U.S. published patent application 2004/0018176 to Reich et al., the entire disclosures of which are herein incorporated by reference.

Expression of a given miRNA can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a miRNA. Preferably, the antisense nucleic acid comprises a nucleic acid sequence that is 50-100% complementary, more preferably 75-100% complementary, and most preferably 95-100% complementary to a contiguous nucleic acid sequence in a miRNA. Without wishing to be bound by any theory, it is believed that the antisense nucleic acids activate RNase H or some other cellular nuclease that digests the miRNA/antisense nucleic acid duplex.

In some embodiments, the miR inhibitor is an antagomir and/or an antisense oligonucleotide.

The term “antagomir” as used herein refers to a chemically engineered small RNA that is used to silence miR-324 or miR-129. The antagomir is complementary to the specific miRNA target with either mis-pairing or some sort of base modification. Antagomirs may also include some sort of modification to make them more resistant to degradation. In some embodiments the antagomir is a chemically engineered cholesterol-conjugated single-stranded RNA analogue.

Inhibition of miR-324 or miR-129 can also be achieved with antisense 2′-O-methyl (2′-O-Me) oligoribonucleotides, 2′-O-methoxyethyl (2′-O-MOE), phosphorothioates, locked nucleic acid (LNA), morpholino oligomers or by use of lentivirally or adenovirally expressed antagomirs (Stenvang and Kauppinen (2008), Expert Opin. Biol. Ther. 8(1):59-81). Furthermore, MOE (2′-O-methoxyethyl phosphorothioate) or LNA (locked nucleic acid (LNA) phosphorothioate chemistry)-modification of single-stranded RNA analogous can be used to inhibit miRNA activity.

Antisense nucleic acids can also contain modifications of the nucleic acid backbone or of the sugar and base moieties (or their equivalent) to enhance target specificity, nuclease resistance, delivery or other properties related to efficacy of the molecule. Such modifications include cholesterol moieties, duplex intercalators such as acridine or the inclusion of one or more nuclease-resistant groups.

Antisense nucleic acids can be produced chemically or biologically, or can be expressed from a recombinant plasmid or viral vector, as described below. Exemplary methods for producing and testing are within the skill in the art; see, e.g., Stein and Cheng (1993), Science 261:1004 and U.S. Pat. No. 5,849,902 to Woolf et al., the entire disclosures of which are herein incorporated by reference.

Expression of a given miRNA can also be inhibited by an enzymatic nucleic acid. As used herein, an “enzymatic nucleic acid” refers to a nucleic acid comprising a substrate binding region that has complementarity to a contiguous nucleic acid sequence of a miRNA, and which is able to specifically cleave the miRNA. Preferably, the enzymatic nucleic acid substrate binding region is 50-100% complementary, more preferably 75-100% complementary, and most preferably 95-100% complementary to a contiguous nucleic acid sequence in a miRNA. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described below. Exemplary methods for producing and testing dsRNA or siRNA molecules are described in Werner and Uhlenbeck (1995), Nucl. Acids Res. 23:2092-96; Hammann et al. (1999), Antisense and Nucleic Acid Drug Dev. 9:25-31; and U.S. Pat. No. 4,987,071 to Cech et al, the entire disclosures of which are herein incorporated by reference.

The miR inhibitor compound of the invention can be obtained using a number of standard techniques. For example, the miR inhibitor compound of the invention can be chemically synthesized or recombinantly produced using methods known in the art. Typically, miR inhibitor compound of the invention are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, 111., USA), Glen Research (Sterling, Va., USA), ChemGenes (Ashland, Mass., USA) and Cruachem (Glasgow, UK).

In some embodiments, of the invention, a synthetic miR inhibitor compound of the invention contains one or more design elements. These design elements include, but are not limited to: (i) a replacement group for the phosphate or hydroxyl of the nucleotide at the 5′ terminus of the complementary region; (ii) one or more sugar modifications. In certain embodiments, a synthetic miR inhibitor compound of the invention has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. In particular embodiments, the sugar modification is a 2′O-Me modification. In further embodiments, there is one or more sugar modifications in the first or last 2 to 4 residues of the complementary region or the first or last 4 to 6 residues of the complementary region.

In some embodiments, the miR inhibitor compound of the invention is resistant to degradation by nucleases. One skilled in the art can readily synthesize nucleic acids which are nuclease resistant, for example by incorporating one or more ribonucleotides that are modified at the 2′-position into the miRNAs. Suitable 2′-modified ribonucleotides include those modified at the 2′-position with fluoro, amino, alkyl, alkoxy, and O-allyl.

Alternatively, the miR inhibitor compound of the invention can be expressed from recombinant linear or circular DNA plasmids using any suitable promoter. Suitable promoters for expressing RNA from a plasmid include, e.g., the U6 promoter sequence, or the cytomegalovirus promoters. Selection of other suitable promoters is within the skill in the art. The recombinant plasmids of the invention can also comprise inducible or regulatable promoters for expression of the miR inhibitor compound of the invention in tumor cells.

The miR inhibitor compound of the invention that is expressed from recombinant plasmids can be isolated from cultured cell expression systems by standard techniques. The miR inhibitor compound of the invention which is expressed from recombinant plasmids can also be delivered to, and expressed directly in, tumor cells. The use of recombinant plasmids to deliver the miR inhibitor compound of the invention to tumor cells is discussed in more detail below.

The miR inhibitor compound of the invention can be expressed from a separate recombinant plasmid, or can be expressed from a unique recombinant plasmid. Preferably, the miR inhibitor compound of the invention is expressed as the nucleic acid precursor molecules from a single plasmid, and the precursor molecules are processed into the functional miR inhibitor compound by a suitable processing system, including processing systems extant within tumor cells. Other suitable processing systems include, e.g., the in vitro Drosophila cell lysate system as described in U.S. published application 2002/0086356 to Tuschl et al. and the E. coli RNAse III system described in U.S. published patent application 2004/0014113 to Yang et al., the entire disclosures of which are herein incorporated by reference.

Selection of plasmids suitable for expressing the miR inhibitor compound of the invention, methods for inserting nucleic acid sequences into the plasmid to express the gene products, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

In some embodiments, a plasmid expressing the miR inhibitor compound of the invention comprises a sequence encoding a miR inhibitor compound precursor under the control of the CMV intermediate early promoter. As used herein, “under the control” of a promoter means that the nucleic acid sequences are located 3′ of the promoter, so that the promoter can initiate transcription of the miR inhibitor compound coding sequences.

The miR inhibitor compound of the invention can also be expressed from recombinant viral vectors. It is contemplated that the miR inhibitor compound of the invention can be expressed from separate recombinant viral vectors, or from a unique viral vector. The miR inhibitor compound expressed from the recombinant viral vectors either can be isolated from cultured cell expression systems by standard techniques or can be expressed directly in tumor cells. The use of recombinant viral vectors to deliver the miR inhibitor compound to tumor cells is discussed in more detail below.

The recombinant viral vectors of the invention comprise sequences encoding the miR-324 or miR-129 inhibitor compound of the invention and any suitable promoter for expressing the miR inhibitor compound sequences. Suitable promoters include, for example, the U6 or HI RNA pol III promoter sequences, or the cytomegalovirus promoters. Selection of other suitable promoters is within the skill in the art. The recombinant viral vectors of the invention can also comprise inducible or regulatable promoters for expression of the miR inhibitor compound in tumor cells.

Any viral vector capable of accepting the coding sequences for the miR inhibitor compound of the invention can be used; for example, vectors derived from adenovirus (AV); adenoassociated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate. For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J. E. et al. (2002), J Virol 76:791801, the entire disclosure of which is herein incorporated by reference.

Selection of recombinant viral vectors suitable for use in the invention, methods for inserting nucleic acid sequences for expressing said miR inhibitor compound of the invention into the vector, methods of delivering the viral vector to the cells of interest, and recovery of the expressed miR inhibitor compound products are within the skill in the art. See, for example, Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are herein incorporated by reference.

Preferred viral vectors are those derived from AV and AAV. A suitable AV vector for expressing the miR inhibitor compound of the invention, a method for constructing the recombinant AV vector, and a method for delivering the vector into target cells, are described in Xia et al. (2002), Nat. Biotech. 20:1006-1010, the entire disclosure of which is herein incorporated by reference. Suitable AAV vectors for expressing the miR inhibitor compound of the invention, methods for constructing the recombinant AAV vector, and methods for delivering the vectors into target cells are described in Samulski et al. (1987), J. Virol. 61:3096-3101; Fisher et al. (1996), J. Virol., 70:520-532; Samulski et al. (1989), J. Virol. 63:3822-3826; U.S. Pat. Nos. 5,252,479; 5,139,941; International Patent Application No. WO 94/13788; and International Patent Application No. WO 93/24641, the entire disclosures of which are herein incorporated by reference. Preferably, the miR inhibitor compound of the invention is expressed from a single recombinant AAV vector comprising the CMV intermediate early promoter.

In some embodiments, a recombinant AAV viral vector of the invention comprises a nucleic acid sequence encoding a miR inhibitor compound precursor in operable connection with a polyT termination sequence under the control of a human U6 RNA promoter. As used herein, “in operable connection with a polyT termination sequence” means that the nucleic acid sequences encoding the sense or antisense strands are immediately adjacent to the polyT termination signal in the 5′ direction. During transcription of the miR inhibitor compound sequences from the vector, the polyT termination signals act to terminate transcription.

In some embodiments, the miR inhibitor consists of a gene editing complex comprising a CRISPR-associated endonuclease and a guide RNA, wherein the guide RNA is complementary to a target nucleic acid sequence within the gene encoding for miR-324 or miR-129.

As used herein, the term “CRISPR-associated endonuclease” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, Mass.).

In some embodiments, the CRISPR-associated endonuclease is a Cpf1 nuclease. As used herein, the term “Cpf1 protein” to a Cpf1 wild-type protein derived from Type V CRISPR-Cpf1 systems, modifications of Cpf1 proteins, variants of Cpf1 proteins, Cpf1 orthologs, and combinations thereof. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237(MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532(7600):517-21 (2016)).

The miR inhibitor compound can be administered to a patient by any means suitable for delivering these compounds to tumor cells. For example, the miR inhibitor compound can be administered by methods suitable to transfect cells of the patient with these compounds, or with nucleic acids comprising sequences encoding these compounds. Preferably, the cells are transfected with a plasmid or viral vector comprising sequences encoding at least one miR inhibitor compound.

The miR inhibitor compound can be administered to a patient by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection (e.g., intra-retinal injection, or subretinal injection); subcutaneous injection or deposition, including subcutaneous infusion (such as by osmotic pumps); direct application to the tissue of interest, for example by a catheter or other placement device (e.g., an implant comprising a porous, non-porous, or gelatinous material); and inhalation. Preferred administration routes are injection, infusion and direct injection into the tumor tissue.

In the present methods, a miR inhibitor compound can be administered to the patient either as naked RNA, in combination with a delivery reagent, or as a nucleic acid (e.g., a recombinant plasmid or viral vector) comprising sequences that express the miR inhibitor compound. Suitable delivery reagents include, e.g, the Minis Transit TKO lipophilic reagent; lipofectin; lipofectamine; cellfectin; polycations (e.g., polyethylenimine which become a nanoparticle (less than 50 nm) when mixed with glucose solution; or e.g, polylysine), and liposomes.

Recombinant plasmids and viral vectors comprising sequences that express the miR inhibitor compounds, and techniques for delivering such plasmids and vectors, are discussed above.

In some embodiments, liposomes are used to deliver a miR inhibitor compound (or nucleic acids comprising sequences encoding them) to a patient. Liposomes can also increase the blood half-life of the gene products or nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream.

A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference. The liposomes for use in the present methods can comprise a ligand molecule that targets the liposome to tumor cells. Ligands which bind to receptors prevalent in tumor cells, such as monoclonal antibodies that bind to cancer cell antigens, are preferred. The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In a particularly preferred embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference. Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; animated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive animation using Na (CN)BH3 and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects will efficiently accumulate these liposomes; see Gabizon, et al. (1988), Proc. Natl. Acad. Sci., USA, 18:6949-53. In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the miR inhibitor compounds (or nucleic acids comprising sequences encoding them) to tumor cells.

One skilled in the art can readily determine a therapeutically effective amount of said compound to be administered to a given patient, by taking into account factors such as the size and weight of the patient; the extent of disease penetration; the age, health and sex of the patient; the route of administration; and whether the administration is regional or systemic. An effective amount of said compound can be based on the approximate or estimated body weight of a patient to be treated. Preferably, such effective amounts are administered parenterally or enterally, as described herein. For example, an effective amount of the compound administered to a patient can range from about 5-10000 micrograms/kg of body weight and is preferably between about 5-3000 micrograms/kg of body weight, and is preferably between about 700-1000 micrograms/kg of body weight, and is more preferably greater than about 1000 micrograms/kg of body weight. One skilled in the art can also readily determine an appropriate dosage regimen for the administration of the compound to a given patient. For example, the compound can be administered to the patient once (e.g., as a single injection or deposition).

In some embodiments, the miR inhibitor of the present invention is administered to the patient in combination with chemotherapy. The term “chemotherapeutic agent” or “chemotherapy agent” are used interchangeably herein and refers to an agent that can be used in the treatment of cancers and neoplasms. In some embodiments, a chemotherapeutic agent can be in the form of a prodrug which can be activated to a cytotoxic form. Chemotherapeutic agents are commonly known by persons of ordinary skill in the art and are encompassed for use in the present invention.

In some embodiments, the miR inhibitor of the present invention is administered to the patient in combination with sorafenib (i.e., sorafenib tosylate as well as other pharmaceutically acceptable forms, salts, and esters of sorafenib). Sorafenib is commercially available as NEXAVAR®, which is the tosylate salt of sorafenib. Sorafenib tosylate has the chemical name 4-(4-{3-[4-Chloro-3 (trifluoromethyl)phenyl]ureido} phenoxy) N-methylpyridine-2-carboxamide 4-methylbenzenesulfonate.

In some embodiments, the miR inhibitor of the present invention is administered to the patient, alone or in combination with IL-27. As uses herein, the term “IL-27” refers herein to a heterodimeric cytokine comprising the subunits p28 and EBI3. Exemplary amino acid sequences of p28 and EBI3 are respectively represented by SEQ ID NO: 5 and SEQ ID NO:6. The term encompasses full-length, unprocessed IL-27 as well as any form of IL-27 that results from processing in the cell or any fragment thereof. The term also encompasses naturally occurring variants of IL-27, e.g., splice variants or allelic variants. In some embodiments, IL-27 is a human IL-27 comprising a p28 (also referred to as IL-27A) having the amino acid sequence of SEQ ID NO:5 and EB13 (also referred to as IL-27B) having the amino acid sequence of SEQ ID NO: 6.

>sp|Q8NEV9|IL27A_HUMAN Interleukin-27 subunit
alpha
OS = Homo sapiens OX = 9606 GN = IL27 PE = 1
SV = 2
SEQ ID NO: 5
MGQTAGDLGWRLSLLLLPLLLVQAGVWGFPRPPGRPQLSLQELRREFTV
SLHLARKLLSEVRGQAHRFAESHLPGVNLYLLPLGEQLPDVSLTFQAWR
RLSDPERLCFISTTLQPFHALLGGLGTQGRWTNMERMQLWAMRLDLRDL
QRHLRFQVLAAGFNLPEEEEEEEEEEEEERKGLLPGALGSALQGPAQVS
WPQLLSTYRLLHSLELVLSRAVRELLLLSKAGHSVWPLGFPTLSPQP
>sp|Q14213|IL27B HUMAN Interleukin-27 subunit
beta
OS = Homo sapiens OX = 9606 GN = EBI3 PE = 1
SV = 2
SEQ ID NO:6
MTPQLLLALVLWASCPPCSGRKGPPAALTLPRVQCRASRYPIAVDCSWT
LPPAPNSTSPVSFIATYRLGMAARGHSWPCLQQTPTSTSCTITDVQLFS
MAPYVLNVTAVHPWGSSSSFVPFITEHIIKPDPPEGVRLSPLAERQLQV
QWEPPGSWPFPEIFSLKYWIRYKRQGAARFHRVGPIEATSFILRAVRPR
ARYYVQVAAQDLTDYGELSDWSLPATATMSLGK

The miR inhibitor compounds of the invention are preferably formulated as pharmaceutical compositions, prior to administering to a patient, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise miR inhibitor compound (e.g., 0.1 to 90% by weight), or a physiologically acceptable salt thereof, mixed with a pharmaceutically-acceptable carrier. The pharmaceutical formulations of the invention can also comprise miR inhibitor compound which are encapsulated by liposomes and a pharmaceutically-acceptable carrier. Preferred pharmaceutically-acceptable carriers are water, buffered water, normal saline, 0.4% saline, 0.3% glycine, hyaluronic acid and the like.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form or can be lyophilized.

### Methods for the Prognosis of Cancer

The first object of the present invention relates to a method for predicting the survival time of a patient suffering from a cancer comprising i) determining the expression level of miR-324 in a sample obtained from the patient, ii) comparing the expression level determined at step i) with a predetermined reference value and wherein a difference between the determine expression level and said predetermined reference value is indicative whether the patient will have a long or short survival time.

In some embodiments, the method is particularly suitable for predicting the survival time of a patient suffering from hepatocellular carcinoma (HCC).

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DFS gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

As used herein, the term “sample” to any biological sample obtained from the purpose of evaluation in vitro.

In some embodiments, the biological sample is a body fluid sample. Examples of body fluids are blood, serum, plasma, amniotic fluid, brain/spinal cord fluid, liquor, cerebrospinal fluid, sputum, throat and pharynx secretions and other mucous membrane secretions, synovial fluids, ascites, tear fluid, lymph fluid and urine. More particularly, the sample is a blood sample. As used herein, the term “blood sample” means a whole blood sample obtained from the patient.

In some embodiments, the sample is a tissue tumor sample. The term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient. In some embodiments, the tumor tissue sample encompasses (i) a global primary tumor (as a whole), (ii) a tissue sample from the centre of the tumor, (iii) lymphoid islets in close proximity with the tumor, (iv) the lymph nodes located at the closest proximity of the tumor, (v) a tumor tissue sample collected prior surgery (for follow-up of patients after treatment for example), and (vi) a distant metastasis. In some embodiments, the tumor tissue sample, encompasses pieces or slices of tissue that have been removed from the tumor, including following a surgical tumor resection or following the collection of a tissue sample for biopsy, for further quantification of several expression level of the miRNA, notably through histology or immunohistochemistry methods, through flow cytometry methods and through methods of gene or protein expression analysis, including genomic and proteomic analysis. The tumor tissue sample can, of course, be patiented to a variety of well-known post-collection preparative and storage techniques (e.g., fixation, storage, freezing, etc.). The sample can be fresh, frozen, fixed (e.g., formalin fixed), or embedded (e.g., paraffin embedded).

According to the invention, measuring the expression level of the miRNA of the invention in the sample obtained from the patient can be performed by a variety of techniques. For example, the nucleic acid contained in the sample is first extracted according to standard methods, for example using lytic enzymes or chemical solutions or extracted by nucleic-acid-binding resins following the manufacturer's instructions. Conventional methods and reagents for isolating RNA from a sample comprise Qiasymphony RNA kit (Qiagen), High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person. The expression level of one or more miRNA in the sample may be determined by any suitable method. Any reliable method for measuring the level or amount of miRNA in a sample may be used. Generally, miRNA can be detected and quantified from a sample (including fractions thereof), such as samples of isolated RNA by various methods known for mRNA, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, in situ hybridization, etc.), and sequencing-based methods (e.g., next-generation sequencing methods, for example, using the Illumina or IonTorrent platforms). Other exemplary techniques include ribonuclease protection assay (RPA) and mass spectroscopy.

In some embodiments, RNA is converted to DNA (cDNA) prior to analysis. cDNA can be generated by reverse transcription of isolated miRNA using conventional techniques. miRNA reverse transcription kits are known and commercially available. Examples of suitable kits include, but are not limited to the mirVana TaqMan® miRNA transcription kit (Ambion, Austin, Tex.), and the TaqMan® miRNA transcription kit (Applied Biosystems, Foster City, Calif.). Universal primers, or specific primers, including miRNA-specific stem-loop primers, are known and commercially available, for example, from Applied Biosystems. In some embodiments, miRNA is amplified prior to measurement. In some embodiments, the expression level of miRNA is measured during the amplification process. In some embodiments, the expression level of miRNA is not amplified prior to measurement. Some exemplary methods suitable for determining the expression level of miRNA in a sample are described in greater hereinafter. These methods are provided by way of illustration only, and it will be apparent to a skilled person that other suitable methods may likewise be used.

Many amplification-based methods exist for detecting the expression level of miRNA nucleic acid sequences, including, but not limited to, PCR, RT-PCR, qPCR, and rolling circle amplification. Other amplification-based techniques include, for example, ligase chain reaction (LCR), multiplex ligatable probe amplification, in vitro transcription (IVT), strand displacement amplification (SDA), transcription-mediated amplification (TMA), nucleic acid sequence-based amplification (NASBA), RNA (Eberwine) amplification, and other methods that are known to persons skilled in the art. A typical PCR reaction includes multiple steps, or cycles, that selectively amplify target nucleic acid species: a denaturing step, in which a target nucleic acid is denatured; an annealing step, in which a set of PCR primers (i.e., forward and reverse primers) anneal to complementary DNA strands, and an elongation step, in which a thermostable DNA polymerase elongates the primers. By repeating these steps multiple times, a DNA fragment is amplified to produce an amplicon, corresponding to the target sequence. Typical PCR reactions include 20 or more cycles of denaturation, annealing, and elongation. In many cases, the annealing and elongation steps can be performed concurrently, in which case the cycle contains only two steps. A reverse transcription reaction (which produces a cDNA sequence having complementarity to a miRNA) may be performed prior to PCR amplification. Reverse transcription reactions include the use of, e.g., a RNA-based DNA polymerase (reverse transcriptase) and a primer. Kits for quantitative real time PCR of miRNA are known, and are commercially available. Examples of suitable kits include, but are not limited to, the TaqMan® miRNA Assay (Applied Biosystems) and the mirVana™ qRT-PCR miRNA detection kit (Ambion). The miRNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence. In some embodiments, custom qRT-PCR assays can be developed for determination of miRNA levels. Custom qRT-PCR assays to measure miRNAs in a sample can be developed using, for example, methods that involve an extended reverse transcription primer and locked nucleic acid modified PCR. Custom miRNA assays can be tested by running the assay on a dilution series of chemically synthesized miRNA corresponding to the target sequence. This permits determination of the limit of detection and linear range of quantitation of each assay. Furthermore, when used as a standard curve, these data permit an estimate of the absolute abundance of miRNAs measured in the samples. Amplification curves may optionally be checked to verify that Ct values are assessed in the linear range of each amplification plot. Typically, the linear range spans several orders of magnitude. For each candidate miRNA assayed, a chemically synthesized version of the miRNA can be obtained and analyzed in a dilution series to determine the limit of sensitivity of the assay, and the linear range of quantitation. Relative expression levels may be determined, for example, according to the 2(-ΔΔ C(T)) Method, as described by Livak et ah, Analysis of relative gene expression data using real-time quantitative PCR and the 2(-ΔΔ C(T)) Method. Methods (2001) December; 25(4):402-8.

In some embodiments, two or more miRNAs are amplified in a single reaction volume. For example, multiplex q-PCR, such as qRT-PCR, enables simultaneous amplification and quantification of at least two miRNAs of interest in one reaction volume by using more than one pair of primers and/or more than one probe. The primer pairs comprise at least one amplification primer that specifically binds each miRNA, and the probes are labeled such that they are distinguishable from one another, thus allowing simultaneous quantification of multiple miRNAs.

Rolling circle amplification is a DNA-polymerase driven reaction that can replicate circularized oligonucleotide probes with either linear or geometric kinetics under isothermal conditions (see, for example, Lizardi et al., Nat. Gen. (1998) 19(3):225-232; Gusev et al, Am. J. Pathol. (2001) 159(0:63-69; Nallur et al, Nucleic Acids Res. (2001) 29(23):E118). In the presence of two primers, one hybridizing to the (+) strand of DNA, and the other hybridizing to the (−) strand, a complex pattern of strand displacement results in the generation of over 109 copies of each DNA molecule in 90 minutes or less. Tandemly linked copies of a closed circle DNA molecule may be formed by using a single primer. The process can also be performed using a matrix-associated DNA. The template used for rolling circle amplification may be reverse transcribed. This method can be used as a highly sensitive indicator of miRNA sequence and expression level at very low miRNA concentrations (see, for example, Cheng et al., Angew Chem. Int. Ed. Engl. (2009) 48(18):3268-72; Neubacher et al, Chembiochem. (2009) 10(8): 1289-91).

miRNA quantification may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values. Many miRNA quantification assays are commercially available from Qiagen (S. A. Courtaboeuf, France), Exiqon (Vedbaek, Denmark) or Applied Biosystems (Foster City, USA).

Expression levels of miRNAs may be expressed as absolute expression levels or normalized expression levels. Typically, expression levels are normalized by correcting the absolute expression level of miRNAs by comparing its expression to the expression of a mRNA that is not a relevant marker for determining whether a patient suffering from acute severe colitis (ASC) will be a responder or a non-responder to a corticosteroid, infliximab and cyclosporine, e.g., a housekeeping mRNA that is constitutively expressed. Suitable mRNAs for normalization include housekeeping mRNAs such as the U6, U24, U48 and S18. This normalization allows the comparison of the expression level in one sample, e.g., a patient sample, to another sample, or between samples from different sources. In a particular embodiment, expression levels are normalized by correcting the absolute expression level of miRNAs by comparing its expression to the expression of a reference mRNA.

Nucleic acids exhibiting sequence complementarity or homology to the miRNAs of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

The probes and primers are “specific” to the miRNAs they hybridize to, i.e. they preferably hybridize under high stringency hybridization conditions (corresponding to the highest melting temperature Tm, e.g., 50 formamide, 5× or 6×SCC. SCC is a 0.15 M NaCl, 0.015 M Na-citrate).

miRNA may be detected using hybridization-based methods, including but not limited to hybridization arrays (e.g., microarrays), NanoString analysis, Northern Blot analysis, branched DNA (bDNA) signal amplification, and in situ hybridization.

Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, inkjet printing, or electrochemistry on microelectrode arrays. Also useful are microfluidic TaqMan Low-Density Arrays, which are based on an array of microfluidic qRT-PCR reactions, as well as related microfluidic qRT-PCR based methods. In one example of microarray detection, various oligonucleotides (e.g., 200+5′-amino-modified-C6 oligos) corresponding to human sense miRNA sequences are spotted on three-dimensional CodeLink slides (GE Health/Amersham Biosciences) at a final concentration of about 20 μM and processed according to manufacturer's recommendations. First strand cDNA synthesized from 20 μg TRIzol-purified total RNA is labeled with biotinylated ddUTP using the Enzo BioArray end labeling kit (Enzo Life Sciences Inc.). Hybridization, staining, and washing can be performed according to a modified Affymetrix Antisense genome array protocol. Axon B-4000 scanner and Gene-Pix Pro 4.0 software or other suitable software can be used to scan images. Non-positive spots after background subtraction, and outliers detected by the ESD procedure, are removed. The resulting signal intensity values are normalized to per-chip median values and then used to obtain geometric means and standard errors for each miRNA. Each miRNA signal can be transformed to log base 2, and a one-sample t test can be conducted. Independent hybridizations for each sample can be performed on chips with each miRNA spotted multiple times to increase the robustness of the data.

Microarrays can be used for the expression profiling of miRNAs. For example, RNA can be extracted from the sample and, optionally, the miRNAs are size-selected from total RNA. Oligonucleotide linkers can be attached to the 5′ and 3′ ends of the miRNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA. Total RNA containing the miRNA extracted from the sample can also be used directly without size-selection of the miRNAs. For example, the RNA can be 3′ end labeled using T4 RNA ligase and a fluorophore-labeled short RNA linker. Fluorophore-labeled miRNAs complementary to the corresponding miRNA capture probe sequences on the array hybridize, via base pairing, to the spot at which the capture probes are affixed. The fluorescence intensity of each spot is then evaluated in terms of the number of copies of a particular miRNA, using a number of positive and negative controls and array data normalization methods, which will result in assessment of the level of expression of a particular miRNA. Several types of microarrays can be employed including, but not limited to, spotted oligonucleotide microarrays, pre-fabricated oligonucleotide microarrays or spotted long oligonucleotide arrays.

Accordingly, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, Brilliant Yellow, coumarin and derivatives such as coumarin, 7-amino-4-methylcoumarin (AMC, Coumarin 120), 7-amino-4-trifluoromethylcoumarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), Dichlorotriazinylamino fluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281 :20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927, 069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlsbad, Calif.).

RT-PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase. The majority of the thermocyclers on the market now offer similar characteristics. Typically, thermocyclers involve a format of glass capillaries, plastics tubes, 96-well plates or 384-well plates. The thermocylcer also involves software analysis.

miRNAs can also be detected without amplification using the nCounter Analysis System (NanoString Technologies, Seattle, Wash.). This technology employs two nucleic acid-based probes that hybridize in solution (e.g., a reporter probe and a capture probe). After hybridization, excess probes are removed, and probe/target complexes are analyzed in accordance with the manufacturer's protocol. nCounter miRNA assay kits are available from NanoString Technologies, which are capable of distinguishing between highly similar miRNAs with great specificity. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each oligonucleotide target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Mass spectroscopy can be used to quantify miRNA using RNase mapping. Isolated RNAs can be enzymatically digested with RNA endonucleases (RNases) having high specificity (e.g., RNase Tl, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESTMS. The presence of post-transcriptional modifications can be revealed by mass shifts from those expected based upon the RNA sequence. Ions of anomalous mass/charge values can then be isolated for tandem MS sequencing to locate the sequence placement of the post-transcriptionally modified nucleoside. Matrix-assisted laser desorption/ionization mass spectrometry (MALDI-MS) has also been used as an analytical approach for obtaining information about post-transcriptionally modified nucleosides. MALDI-based approaches can be differentiated from EST-based approaches by the separation step. In MALDI-MS, the mass spectrometer is used to separate the miRNA. To analyze a limited quantity of intact miRNAs, a system of capillary LC coupled with nanoESl-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR® XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact miRNAs are eluted from the trap column and directly injected into a CI 8 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for miRNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple miRNAs can be detected in a microarray format using a novel approach that combines a surface enzyme reaction with nanoparticle-amplified SPR imaging (SPRI). The surface reaction of poly(A) polymerase creates poly(A) tails on miRNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for miRNA profiling at attamole levels. miRNAs can also be detected using branched DNA (bDNA) signal amplification (see, for example, Urdea, Nature Biotechnology (1994), 12:926-928). miRNA assays based on bDNA signal amplification are commercially available. One such assay is the QuantiGene® 2.0 miRNA Assay (Affymetrix, Santa Clara, Calif.). Northern Blot and in situ hybridization may also be used to detect miRNAs. Suitable methods for performing Northern Blot and in situ hybridization are known in the art. Advanced sequencing methods can likewise be used as available. For example, miRNAs can be detected using Illumina® Next Generation Sequencing (e.g. Sequencing-By-Synthesis or TruSeq methods, using, for example, the HiSeq, HiScan, GenomeAnalyzer, or MiSeq systems (Illumina, Inc., San Diego, Calif.)). miRNAs can also be detected using Ion Torrent Sequencing (Ion Torrent Systems, Inc., Gulliford, Conn.), or other suitable methods of semiconductor sequencing.

In some embodiments, the expression level of the miRNA is determined by RNA-seq. As used, the term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq typically includes whole transcriptome sequencing. As used herein, the term “whole transcriptome sequencing” refers to the use of high throughput sequencing technologies to sequence the entire transcriptome in order to get information about a sample's RNA content. Whole transcriptome sequencing can be done with a variety of platforms for example, the Genome Analyzer (Illumina, Inc., San Diego, Calif.) and the SOLiD™ Sequencing System (Life Technologies, Carlsbad, Calif.), However, any platform useful for whole transcriptome sequencing may be used. Typically, the RNA is extracted, and ribosomal RNA may be deleted as described in U.S. Pub, No. 2011/0111409. cDNA sequencing libraries may be prepared that are directional and single or paired-end using commercially available kits such as the ScriptSeg™ M mRNA-Seq Library Preparation Kit (Epicenter Biotechnologies, Madison, Wis.). The libraries may also be barcoded for multiplex sequencing using commercially available barcode primers such as the RNA-Seq Barcode Primers from Epicenter Biotechnologies (Madison, Wis.). PCR is then carried out to generate the second strand of cDNA to incorporate the barcodes and to amplify the libraries. After the libraries are quantified, the sequencing libraries may be sequenced. Nucleic acid sequencing technologies are suitable methods for expression analysis. The principle underlying these methods is that the number of times a (DNA sequence is detected in a sample is directly related to the relative RNA levels corresponding to that sequence. These methods are sometimes referred to by the term Digital Gene Expression (DOE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). Typically RNA-seq uses Next Generation Sequencing or NGS. As used herein, the term “Next Generation Sequencing” (NGS) refers to a relatively new sequencing technique as compared to the traditional Sanger sequencing technique. For review, see Shendure et al., Nature Biotech., 26(10): 1135-45 (2008), which is hereby incorporated by reference into this disclosure. For purpose of this disclosure, NGS may include cyclic array sequencing, microelectrophoretic sequencing, sequencing by hybridization, among others. By way of example, in a typical NGS using cyclic-array methods, genomic DNA or cDNA library is first prepared, and common adaptors may then be ligated to the fragmented genomic DNA or cDNA. Different protocols may be used to generate jumping libraries of mate-paired tags with controllable distance distribution. An array of millions of spatially immobilized PCR colonies or “polonies” is generated with each polonies consisting of many copies of a single shotgun library fragment. Because the polonies are tethered to a planar array, a single microliter-scale reagent volume can be applied to manipulate the array features in parallel, for example, for primer hybridization or for enzymatic extension reactions. Imaging-based detection of fluorescent labels incorporated with each extension may be used to acquire sequencing data on all features in parallel. Successive iterations of enzymatic interrogation and imaging may also be used to build up a contiguous sequencing read for each array feature.

In some embodiments, the method of the present invention further comprises the steps of i) determining the expression level of WSX-1 and ii) comparing the determined expression level with a corresponding predetermined value.

As used herein, the term “WSX-1” has its general meaning in the art and refers to the interleukin-27 receptor subunit alpha. The term is also known as L27RA, CRL1, or TCCR. An exemplary amino acid sequence for WSX-1 is represented by SEQ ID NO:7.

>sp|Q6UWE1|I27RA_HUMAN Interleukin-27 receptor
subunit
alpha OS = Homo sapiens OX = 9606 GN = IL27RA
PE = 2 SV = 2
SEQ ID NO: 7
MRGGRGAPFWLWPLPKLALLPLLWVLFQRTRPQGSAGPLQCYGVGPLGD
LNCSWEPLGDLGAPSELHLQSQKYRSNKTQTVAVAAGRSWVAIPREQLT
MSDKLLVWGTKAGQPLWPPVEVNLETQMKPNAPRLGPDVDFSEDDPLEA
TVHWAPPTWPSHKVLICQFHYRRCQEAAWTLLEPELKTIPLTPVEIQDL
ELATGYKVYGRCRMEKEEDLWGEWSPILSFQTPPSAPKDVWVSGNLCGT
PGGEEPLLLWKAPGPCVQVSYKVWFWVGGRELSPEGITCCCSLIPSGAE
WARVSAVNATSWEPLTNLSLVCLDSASAPRSVAVSSIAGSTELLVTWQP
GPGEPLEHVVDWARDGDPLEKLNWVRLPPGNLSALLPGNFTVGVPYRIT
VTAVSASGLASASSVWGFREELAPLVGPTLWRLQDAPPGTPAIAWGEVP
RHQLRGHLTHYTLCAQSGTSPSVCMNVSGNTQSVTLPDLPWGPCELWVT
ASTIAGQGPPGPILRLHLPDNTLRWKVLPGILFLWGLFLLGCGLSLATS
GRCYHLRHKVLPRWVWEKVPDPANSSSGQPHMEQVPEAQPLGDLPILEV
EEMEPPPVMESSQPAQATAPLDSGYEKHFLPTPEELGLLGPPRPQVLA

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the score in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the score in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples; b) providing, for each sample provided at step a), information relating to the actual clinical outcome for the corresponding patient (i.e. the duration of the survival); c) providing a serial of arbitrary quantification values; d) determining the expression level of the miRNA for each sample contained in the collection provided at step a) so as to calculate the score as described above; e) classifying said samples in two groups for one specific arbitrary quantification value provided at step c), respectively: (i) a first group comprising samples that exhibit a quantification value for the score that is lower than the said arbitrary quantification value contained in the said serial of quantification values; (ii) a second group comprising samples that exhibit a quantification value for said score that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the patients from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example, the score has been assessed for 100 samples of 100 patients. The 100 samples are ranked according to the determined score. Sample 1 has the highest score and sample 100 has the lowest score. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patient, Kaplan Meier curves are prepared for each of the 99 groups of two subsets. Also, for each of the 99 groups, the p value between both subsets was calculated. The predetermined reference value is then selected such as the discrimination based on the criterion of the minimum p value is the strongest. In other terms, the score corresponding to the boundary between both subsets for which the p value is minimum is considered as the predetermined reference value.

In some embodiments, the predetermined reference value thus allows discrimination between a poor and a good prognosis for a patient. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the expression level, where values higher than 5 reveal a poor prognosis and values less than 5 reveal a good prognosis. In some embodiments, a patient may be assessed by comparing values obtained by measuring the expression level and comparing the values on a scale, where values above the range of 4-6 indicate a poor prognosis and values below the range of 4-6 indicate a good prognosis, with values falling within the range of 4-6 indicating an intermediate occurrence (or prognosis).

In some embodiments, when the expression level of the miRNA (i.e. miR-324) is higher than the predetermined reference value, it is concluded that the patient will have a short survival time (“poor prognosis”). On the contrary, when the expression level of the miRNA (i.e. miR-324) is lower than the predetermined reference value, it is concluded that the patient will have a long survival time (“good prognosis”). Thus, increased expression level of miR-324 correlates with a short survival time. Moreover, by combining determination of expression level of WSX-1, increased expression level of miR-324 combined to a decreased expression level of WSX-1 correlate with a short survival time.

In some embodiment, in view of the currently limited options for cancer management, the group of biomarkers as disclosed herein is useful for identifying patients with poor-prognosis, in particular patients that are likely to metastasize.

Accordingly, a patient identified with a poor prognosis can be administered with a particular therapy. In some embodiments, a patient identified with a poor prognosis can be administered with a miR inhibitor as described above.

In some embodiments, the method of the present invention be used to identify patients in need of frequent follow-up by a physician or clinician to monitor cancer disease progression. Screening patients for identifying patients having a poor prognosis is also useful to identify patients most suitable or amenable to be enrolled in clinical trial for assessing a therapy for cancer, which will permit more effective subgroup analyses and follow-up studies. Furthermore, the expression level herein can be monitored in patients enrolled in a clinical trial to provide a quantitative measure for the therapeutic efficacy of the therapy which is patient to the clinical trial.

This invention also provides a method for selecting a therapeutic regimen or determining if a certain therapeutic regimen is more appropriate for a patient identified as having a poor prognosis as identified by the methods as disclosed herein. For example, an aggressive anti-cancer therapeutic regime can be perused in which a patient having a poor prognosis, where the patient is administered a therapeutically effective amount of an anti-cancer agent to treat the cancer. In some embodiments, a patient can be monitored for cancer using the methods and biomarkers as disclosed herein, and if on a first (i.e. initial) testing the patient is identified as having a poor prognosis, the patient can be administered an anti-cancer therapy, and on a second (i.e. follow-up testing), the patient is identified as having a good prognosis, the patient can be administered an anti-cancer therapy at a maintenance dose. The method of the present invention is particularly suited to determining which patients will be responsive or experience a positive treatment outcome to a treatment. In general, a therapy is considered to “treat” cancer if it provides one or more of the following treatment outcomes: increase median survival time or decrease metastases. In some embodiments, an anti-cancer therapy is, for example but not limited to administration of a chemotherapeutic agent, radiotherapy etc. Such anti-cancer therapies are disclosed herein, as well as others that are well known by persons of ordinary skill in the art and are encompassed for use in the present invention.

The invention will be further illustrated by the following figures and examples. However, these examples and figures should not be interpreted in any way as limiting the scope of the present invention.

### FIGURES

FIG. 1. Impact of WSX-1 expression on disease-free survival (DFS) and overall survival (OS) in HCC patients.

Percentage of disease-free survival or overall survival in HCC patients has been analyzed according to WSX-1 strong (high, n=40) or low (n=71) expression in tumors. Log-rank test has been used to determine statistical significance (p-Value). A univariate Cox model was used to estimate HR and CI95%. Abbreviations: HR, hazard ratio; CI95%, confidence interval (95%); HCC, hepatocellular carcinoma.

FIG. 2. WSX-1 lack of expression correlates with high tumor proliferation in human HCC.

Percentage of WSX-1 strong or weak expression in HCC patients with high (MIB-^{high}) or low (MIB-1^{low}) tumor proliferation. Chi square test has been used to determine statistical significance.

FIG. 3. IL-27 inhibits tumor cell expansion in vitro in HepG2 but not in Hep3B cell line.

A. Quantification of proliferating cells in HepG2 and Hep3B cells was determined using a Ki-67 immunostaining after 24, 48, or 72 hours treatment with IL-27 (50 ng/mL) or not. B. HepG2 and Hep3B proliferation was assessed using spheroid diameter measurement at 7, 13, 17 and 20 days of culture in low adherence conditions, with or without IL-27 treatment (50 ng/mL). **P<0.01, ***P<0.001 untreated vs IL-27 treated. Data represent mean+/−SEM.

FIG. 4. WSX-1 expression is decreased in HCC lines.

Flow cytometric histograms showing mean fluorescence intensity (MFI) levels of WSX-1 expression in Primary Human Hepatocytes (PHH) compared to HepG2, and Hep3B cell lines.

FIG. 5. Identification of microRNAs leading to the loss of WSX-1 expression in HCC.

Validation of selected microRNA candidates in HCC lines. Absolute quantification of mir-129 and mir-324 was performed by qRT-PCR in primary human hepatocytes (PHH), in HepG2, and Hep3B cells. **P<0.01, ***P<0.001; PHH vs HCC lines; HepG2 vs Hep3B cells. Data represent mean+/−SEM. Abbreviations: qRT-PCR, quantitative reverse-transcription polymerase chain reaction.

FIG. 6. Inhibition of mir-324 and mir-129 restores WSX-1 expression and IL-27 anti-proliferative effects on Hep3B cells in vitro.

Immunocytofluorescence labelling for WSX-1 expression was performed on Hep3B cells treated with negative control antagomirs (25 nM), antagomir-324 (25 nM) or antagomir-129 (25nM). Nuclei were counterstained with DAPI.C. Hep3B proliferation was assessed using spheroid diameter measurement at 7, 13, 17 and 20 days of culture in low adherence condition, with or without IL-27 treatment (50 ng/mL), antagomir-324 (25 nM) and antagomir-129 (25 nM). **P<0.01, ***P<0.001 untreated vs IL-27 treated. Data represent mean+/−SEM. Abbreviations: A-129, antagomir-129; A-324, antagomir-324.

FIG. 7. Mir-324 overexpression in HCC-patients is associated with a higher risk of HCC recurrence.

A) Validation of selected microRNA candidates in samples from HCC patients. Absolute quantification of mir-129 and mir-324 was assessed by qRT-PCR in HCC patients. *P<0.05, **P<0.01; Healthy vs WSX-1^{low} and WSX-1^{high} patients. Data represent mean+/−SEM. B) Percentage of disease-free survival (DFS) of HCC patients was determined according to mir-129 strong (high, n=31) or low (n=37) expression, and C) according to mir-324 high (n=25) or low (n=25) expression in tumors. D) DFS was also determined according to both WSX-1 and mir-324 expression in HCC patients (n=68) Log-rank test has been used to determine statistical significance (p-Value). A univariate Cox model was used to estimate HR and CI95%.

FIG. 8. Experimental design for antagomir-324-5p with or without IL-27 treatments in chronic DEN induced-HCC murine model.

C57Bl/6 mice were subjected to a first injection of DEN (25 mg/kg) at 2 weeks after birth. From 5 weeks, DEN was chronically administered twice a week for 14 weeks (10 mg/kg). Four weeks prior sacrifice, mice were treated or not, twice a week with A324 (5 mg/kg, intravenously). Mice were also subcutaneously implanted with an osmotic pump for the delivery of vehicle or IL-27 for the last 4 weeks. Abbreviations: DEN: diethylnitrosamine; A324: antagomir-324-5p.

FIG. 9. Impact of antagomir-324-5p and IL-27 treatments on chronic DEN-induced HCC murine model.

A. Follow-up of mouse body weight along with chronic DEN administration. B. Analysis of liver weight over body ratio according to the treatments. C. WSX-1 protein expression analysis after immunohistochemistry and semi-quantitative score (0: no expression to 3: highest intensity of staining). Data represent mean+/−SEM. Abbreviations: DEN: diethylnitrosamine; CT neg Antagomir: Antagomir Negative control; A324: antagomir-324-5p

FIG. 10. Impact of antagomiR-324-5p and IL-27 treatments on tumor and fibrosis development in chronic DEN-induced HCC murine model.

A. Analysis of mRNA expression of HCC markers by qPCR in the livers from DEN-injected mice, treated or not with IL-27 and/or A324. B. Analysis of Red Sirius expression by immunostaining using a semiquantitative score, and of fibrosis marker expressions by qPCR in DEN-injected mice treated or not with IL-27 and/or A324. Data represent mean+/−SEM. Abbreviations: DEN: diethylnitrosamine; PCR: polymerase chain reaction; CT neg Antagomir: Antagomir Negative control; A324: antagomir-324-5p.

### METHODS

### Patients and Samples

A total of 114 tumors samples from resection-treated hepatocellular carcinoma (HCC) patients were included in the study. All these patients did not receive HCC treatment before the surgery. Frozen and formalin-fixed paraffin-embedded (FFPE) samples of tumors from resected HCCs were given by the Pathology Department from Henri Mondor University Hospital (Creteil, France). The local ethics committee Ile de France approved the study as required by French legislation.

### Immunohistochemistry on Tumor Specimen

WSX-1 immunostaining. WSX-1 expression was assessed by immunohistochemistry on paraffin-embedded sections of HCC tumors using an anti-WSX-1 antibody (Novus). Hematoxylin was used to counterstain nucleus. To determine physiological expression of WSX-1, 3 first slides of non-pathological livers were studied, and a first review of a 50 HCC slides training set was performed by two different scientists, including a specialized pathologist in liver diseases. Then, the entire cohort was analyzed by the two scientists, independently, using a semiquantitative score which divided patients into two groups: low versus high density of stained cells. WSX-1 expression was considered as high if stained tumor cells showed a strong intensity staining and if number of tumor stained cells are greater than 50%.

MIB-1 expression. MIB-1 immunohistochemistry was automatically performed at the Henri Mondor University hospital (Créteil, France) Pathology Department on Leica Bondmax automat and using an anti-MIB-1 antibody (Sigma Aldrich). MIB-1 proliferation index was considered high if stained cells are greater than 10-positive cells per field (×400).

### Cell Lines

HCC lines were maintained in DMEM medium supplemented with 10% fetal bovine serum (FBS), 100 U/mL of penicillin/streptomycin (PS) and 4 mM glutamine, at 37° C. in a humidified atmosphere containing 5% CO₂.

### IL-27 Anti-Proliferative Effects Assay

Ki-67. For Ki-67 immunofluorescent staining on HCC lines, cells were treated or not with recombinant human IL-27 (rhIL-27, R&D systems) for 24, 48 or 72 hours. Cells were fixed in 2% paraformaldehyde (PFA), then permeabilized with 0.3% Triton X-114. Ki-67 immunostaining was performed using an anti-Ki-67 monoclonal antibody (LifeTechnologies). Nuclei were counterstained with DAPI. Ki-67 positive cells number per field (×200) was counted using the ImageJ software.

Spheres. Cells were seeded in 24 wells low cell-attachment surface plates in DMEM-F12 medium supplemented with 100 U/mL PS, 1× B27 complement, 20 ng/mL recombinant human basic fibroblast growth factor (rhbFGF), and 20 ng/mL recombinant human epidermal growth factor (rhEGF). Cells were treated or not with rhIL-27 for 21 days. Spheres diameters were measured using the ImageJ software.

### WSX-1 Expression Analysis

Flow Cytometry. HCC lines were incubated with an AlexaFluor488 anti-WSX-1 antibody (Novus). WSX-1 expression was analyzed on a Cyan ADP flow cytometer (Beckman Coulter) using FACSDiva software (BD Biosciences). Overlays were built by using FlowJo software.

Immunocytochemistry. Cells were fixed with methanol and blocking of unspecific sites were performed with PBS/1% BSA solution. WSX-1 immunocytofluorescent staining on HCC lines, was performed thanks to an anti-WSX-1 antibody (Novus). Nuclei were counterstained with DAPI and cover-slipped.

### Prediction of MicroRNAs Targeting WSX-1 and Identification of Overexpressed MicroRNAs in HCC

Search for predicted miRNAs targeting WSX-1 was done using three online software including miRanda (from Memorial Sloan-Kettering Cancer Center), TargetScan Human (from Whitehead Institute for Biomedical Research), and MicroCosm Targets (developed by the Enright Lab at European Bioinformatic Institute). miRNA expression profiles in HCC patients and in various HCC lines were retrieved from the National Center for Biotechnology Information (NCBI) using the Gene Expression Omnibus (GEO) database. Then, microarray data were analyzed has been performed in the following eligible datasets: GSE74618, GSE57555, GSE31164, GSE74618, GSE20077, GSE71107 and GSE41077, to identify differentially expressed miRNAs using GEO2R tool in the GEO database. Differentially expressed miRNAs were screened using an adjusted p-value (adj. P) of less than 0.05 and a fold change of at least 1.5 (>1.5-FC) as thresholds. The Venn's diagram was used to match the predicted miRNA targeting WSX-1 and those upregulated in HCC patients, and HCC cell lines. A total of 4 candidate miRNAs (miR-324, miR-129, miR-371, miR-140) were selected for further investigation.

### MicroRNA Detection and Absolute Quantification

Total RNA was extracted from HCC lines and from frozen tumors of HCC patients and a total of 10 ng of RNA was used to cDNA preparation using miScript RT kit (Qiagen). Detection of miR-129 and mir-324 was performed by qRT-PCR on a LightCycler480 (Roche), using the miScript SYBR Green PCR kit (Qiagen), following manufacturer's instructions. microRNA expression was determined as absolute quantification. To determine the absolute number of miR-129 and miR-324 copies, standard range was established for each microRNA with 10 dilutions (from 1.36×10″ to 1.36×10⁵ copies of miRNA) of miR-129 and miR-324 mimics (Dharmacon). The standard curves were established by picking up Ct values using a semi-logarithmic scale and were used for determining the miRNA copy numbers in each unknown sample.

### Oligonucleotide Transfections

Antagomir-129, antagomir-324, and antagomir-negative control (all purchased at Dharmacon) were transfected in Hep3B cells according to manufacturer's instruction. Dy547 labeled antagomir-negative control has been used to evaluate transfection efficiency. Transfected cells were maintained at least for 3 days after transfection.

### Statistical Analysis

GraphPad Prism software was used to perform statistical analysis. The results are expressed as mean+/−SEM. For analyzed data obtained from patients, values were compared using Chi square test. A Kaplan-Meier analysis was performed for disease-free and overall survival rate with the log-rank test (Mantel-Cox). For WSX-1, mir-129 and mir-324 survival curves, analysis has been performed according their low or high expression. For in vitro and PCR studies, statistical significance between two groups was determined by Student t test. Significant difference of data was considered for P<0.05.

### EXAMPLE 1

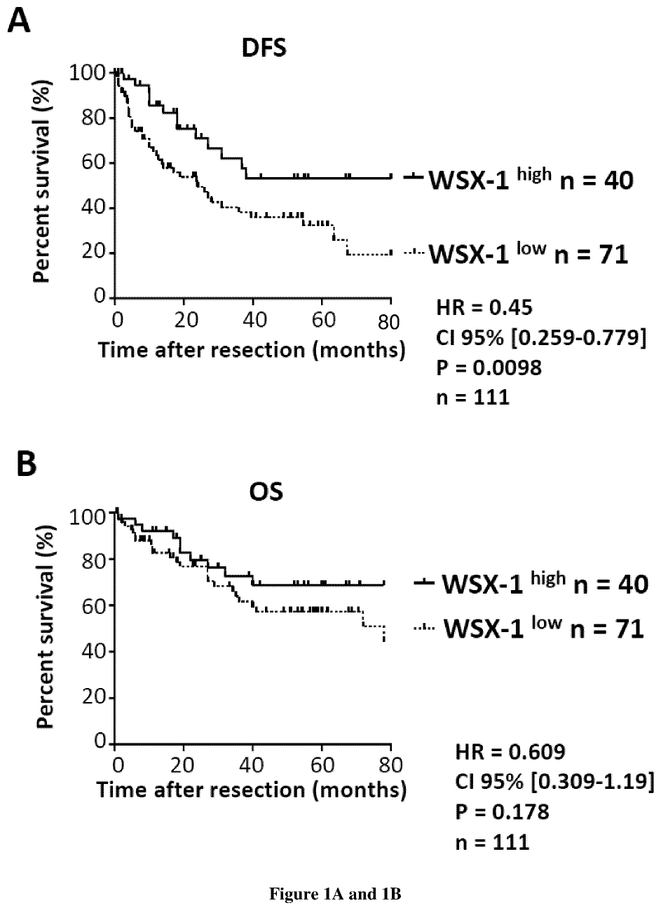

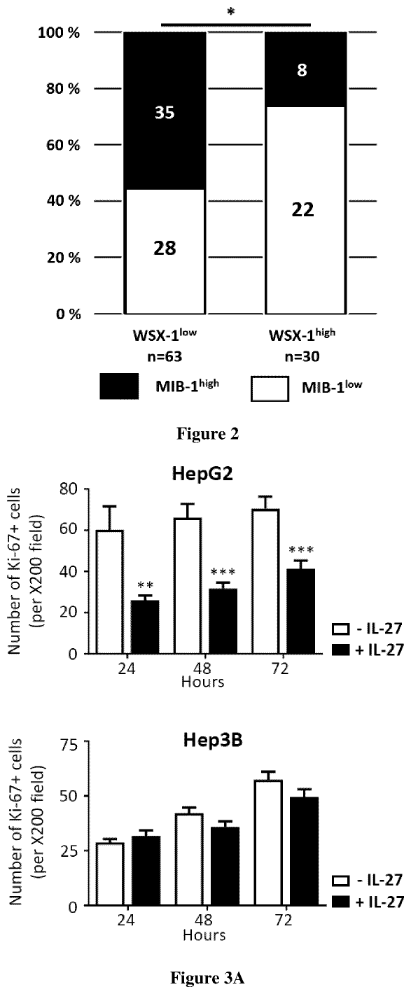

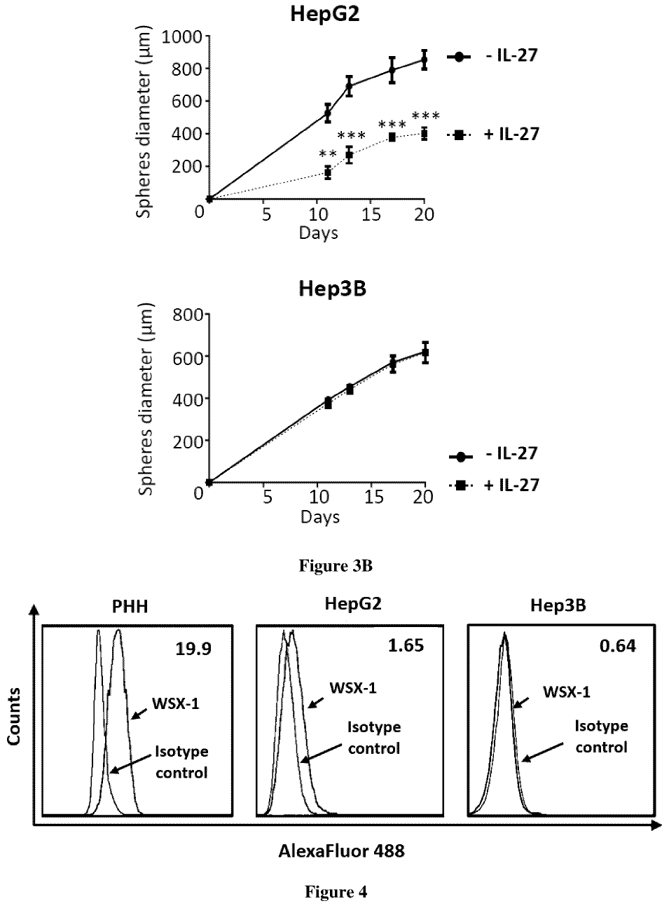

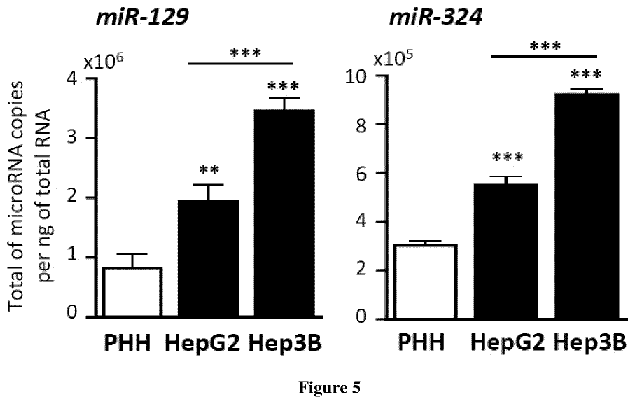

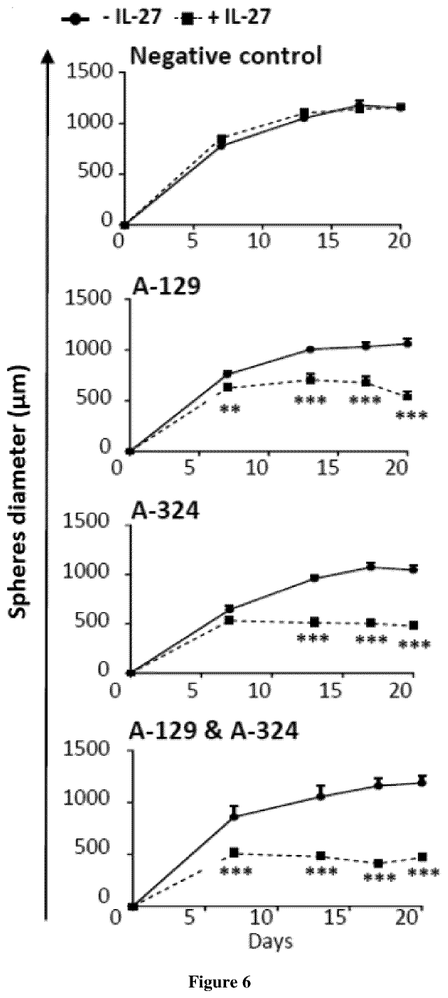

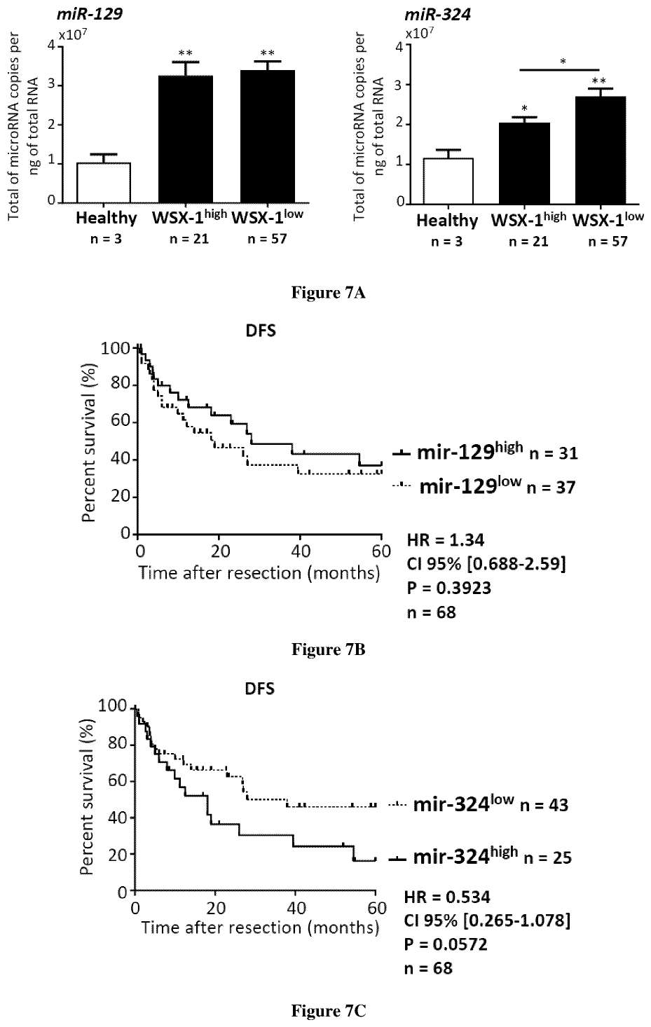

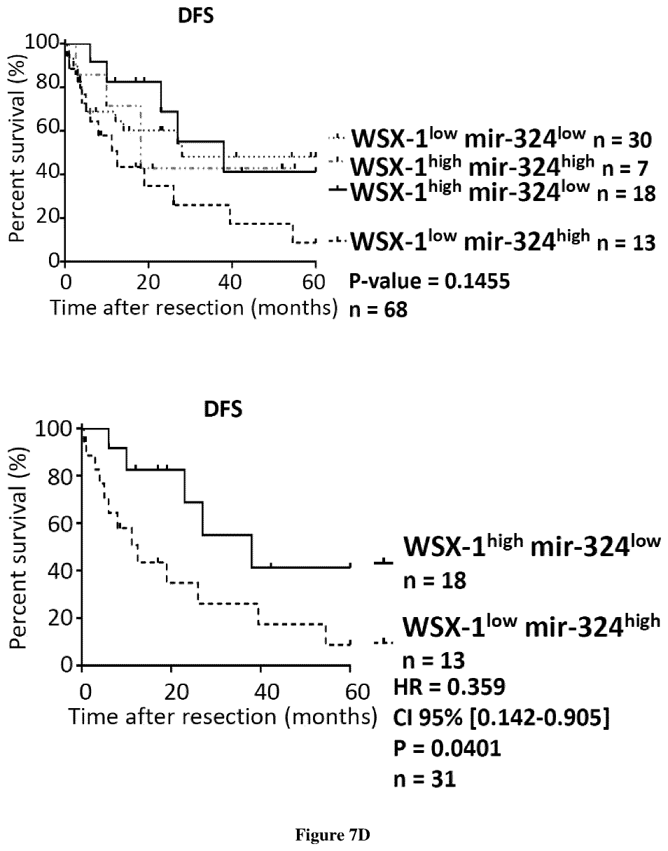

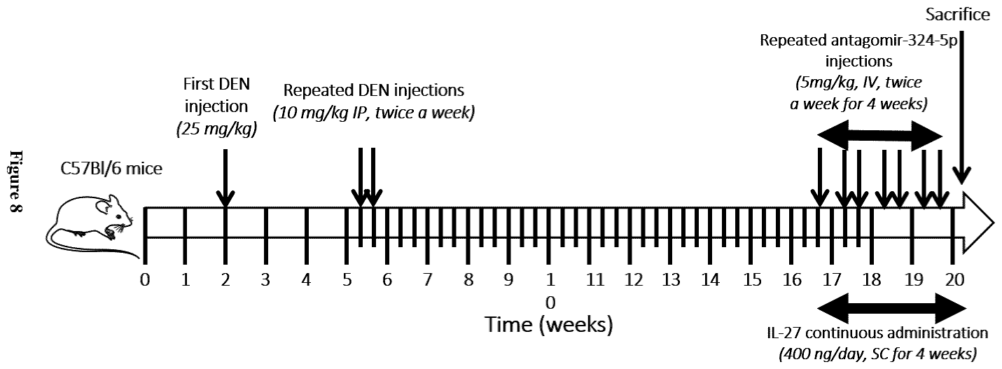

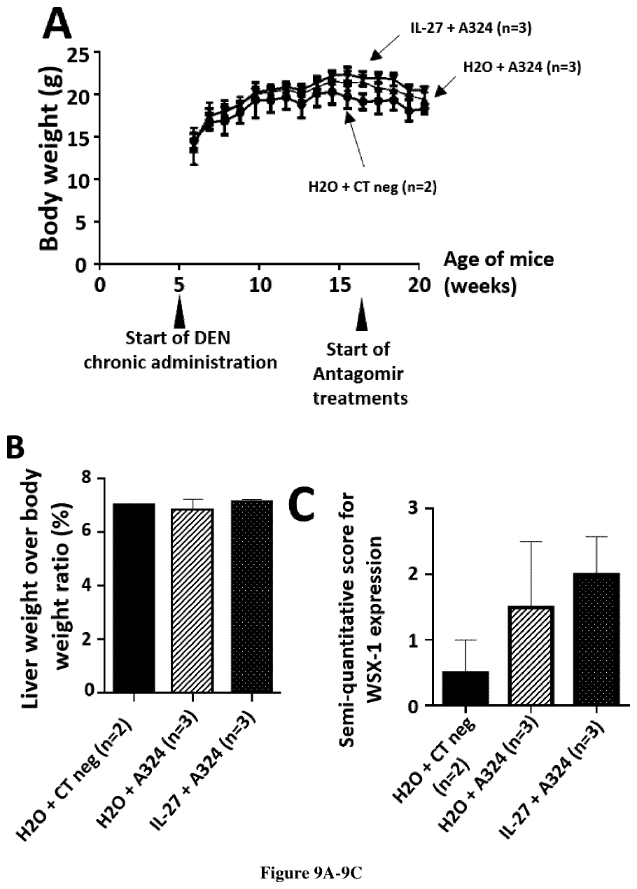

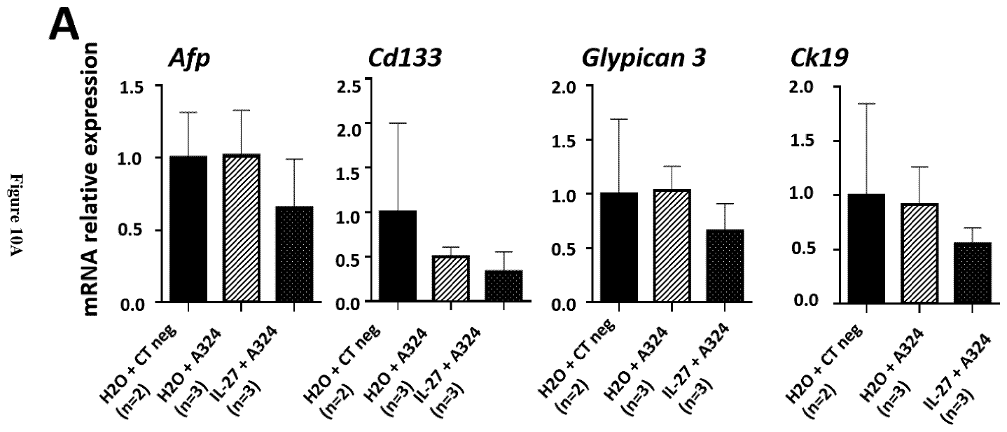

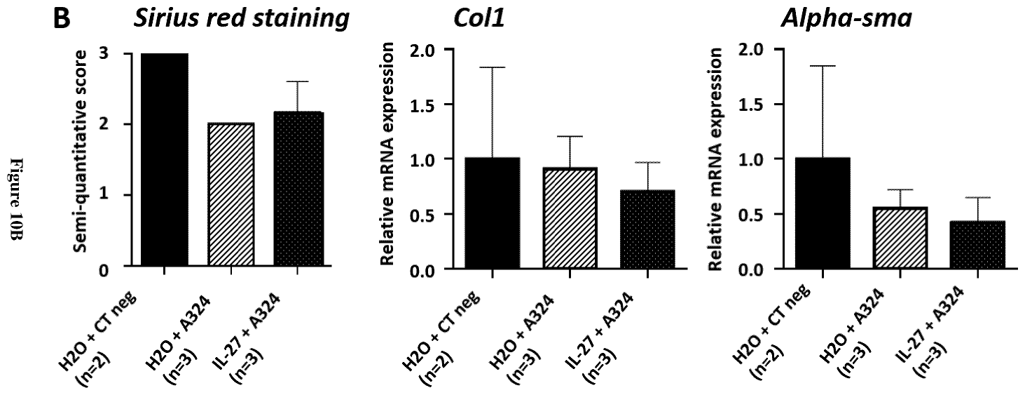

Hepatocellular carcinoma (HCC) has become the most common primary hepatic malignancy. Current therapies are now satisfying and there is therefore an important need for identifying new therapeutic avenues. IL-27 is a cytokine produced in liver microenvironment but its role in the pathogenesis of HCC has never been investigated. The inventors now show that IL-27 exerts anti-proliferative activities in HCC cell lines. However, the inventors show that in patients suffering from HCC that a decreased expression of WSX-1 (i.e. the IL-27 receptor) is associated with a worse prognosis and contributes to the tumor proliferation. The inventors then identified some microRNAs (miR) that are capable of repressing the expression of WSX-1 and show that overexpression of said miR are associated with a worse prognosis in patients. Finally, the inventors demonstrate that antagomirs restore the expression of WSX-1 that can thus restore the tumor cell sensitization to IL-27 properties. More particularly, the results are depicted in Table 1 as well as FIGS. 1-7.

TABLE 1
Association of WSX-1 expression with HCC prognostic factors. Correlation
between WSX-1 strong (high, n = 72) or low (n = 42) expression in tumors with
different HCC prognostic factors has been studied using Chi square test. Correlated
parameters are highlighted in bold. Abbreviations: AFP, Alphafoetoprotein.

| | WSX-1^{low} | WSX-1^{high} | |
|---|---|---|---|
| Parameters | (n = 72) | (n = 42) | p-value |
| Gender (Male)-n (%) | 61 (87) | 36 (82) | 0.43 |
| AFP > 20 ng/mL-n (%) | 26 (44) | 16 (39) | 0.83 |
| Cirrhosis (F4)-n (%) | 25 (39) | 16 (41) | >0.99 |
| Satellites nodules-n (%) | 30 (43) | 12 (27) | 0.11 |
| Microvascular invasion-n (%) | 41 (59) | 14 (33) | 0.007 |
| Macrovascular invasion-n (%) | 20 (29) | 3 (7) | 0.007 |
| Tumor necrosis-n (%) | 37 (56) | 13 (34) | 0.04 |
| Edmondson-Steiner Grade III-IV-n (%) | 48 (69) | 10 (41) | <0.0001 |

### EXAMPLE 2

FIG. 8 shows the Experimental design for antagomir-324-5p with or without IL-27 treatments in chronic DEN induced-HCC murine model. DEN Chronic treatment leads to a sharp decrease in WSX-1 expression in livers. Antagomir-324-5p treatment with or without IL-27 allowed to partially restore WSX-1 protein expression (FIGS. 9A to 9C). Antagomir-324-5p treatment with or without IL-27 allowed to reduce HCC mRNA marker expressions. Antagomir-324-5p treatment with or without IL-27 led to reduced fibrosis related-gene marker expressions and sirius red staining in DEN-injected mice. (FIGS. 10A and 10B).

### REFERENCES

Throughout this application, various references describe the state of the art to which this invention pertains. The disclosures of these references are hereby incorporated by reference into the present disclosure.

1. A method of treating cancer in patient in need thereof comprising administering to the patient a therapeutically effective amount of a miR-324 inhibitor and/or miR-129 inhibitor.

2. The method of claim 1 wherein the cancer is hepatocellular carcinoma (HCC).

3. The method of claim 1 wherein the miR-324 inhibitor and/or miR-129 inhibitor is a nucleic acid that hybridizes with miR-324 or miR-129 or which has a sequence complementarity to that of miR-324 or miR-129.

4. The method of claim 1 wherein the miR-324 inhibitor and/or miR-129 inhibitor is selected from the group consisting of double-stranded RNAs, antagomirs, antisense nucleic acids, enzymatic RNA molecules, and gene editing systems comprising a CRISPR-associated endonuclease and a guide RNA, wherein the guide RNA is complementary to a target nucleic acid sequence within the gene encoding for miR-324 or miR-129.

5. The method of claim 1 wherein the miR-324 inhibitor and/or miR-129 inhibitor is administered to the patient in combination with IL-27.

6. A method for predicting the survival time of and treating a patient suffering from a cancer comprising i) determining the expression level of miR-324 in a sample obtained from the patient, and ii) administering to the subject a therapeutically effective amount of an miR-324 inhibitor and/or miR-129 inhibitor when the expression level of the miR-324 is higher than a predetermined reference value.

7. The method of claim 6 wherein the patient is identified as having a poor prognosis.

8. The method of claim 6 wherein the cancer is hepatocellular carcinoma (HCC).

9. The method of claim 6 which further comprises the steps of i) determining the expression level of WSX-1 and ii) comparing the expression level with a corresponding predetermined value.

10. The method of claim 6 wherein when the expression level of the miR-324 is higher than a predetermined reference value, it is concluded that the patient will have a short survival time.

11. The method of claim 6 wherein when the expression level of miR-324 is lower than a predetermined reference value, it is concluded that the patient will have a long survival time.

12. (canceled)

13. The method of claim 4, wherein the double-stranded RNAs are short- or small-interfering RNAs (siRNAs).

14. The method of claim 4, wherein the enzymatic RNA molecules are ribozymes.
